(19) United States
(12) Patent Application Publication
(10) Pub. No.: US 2008/0292629 A1
(43) Pub. Date: Nov. 27, 2008

(54) System Clock Generation Circuit

(76) Inventors: Yhengbin Yao, Sugar Land, TX (US); Guanghui Hu, Phoenixville, PA (US); Shen-Wu Wang, Sugar Land, TX (US)

Correspondence Address: Cheryl Liljestrand;Tanox, 10301 Stella Link Road, Houston, TX, 77025, US

(21) Appl. No.: 11/597,769

(22) PCT Filed: May 26, 2005
(86) PCT No.: PCT/US05/18642

§ 371 Date: Jan. 4, 2008

Related U.S. Application Data
(60) Provisional application No. 60/574,734, filed on May 27, 2004.

(51) Int. Cl. A61K 39/395; C12N 5/06; C12Q 1/02; G01N 33/566; A61P 11/06

(52) U.S. Cl. 424/138.1; 435/375; 435/29; 435/7.21

(57) ABSTRACT

A system clock signal generator circuit comprising a first PLL circuit that is frequency and phase locked to a wobble signal; a frequency and phase comparator for comprising a first output signal from the first PLL circuit with a system clock signal as frequency divided by M and for outputting a second output signal based on the differences in frequency and in phase; a PLL filter for providing a predetermined cutoff to the second output signal to output a third output signal; a pulse width modulating circuit for generating a pulse wave, the carrier frequency of which is a second reference clock signal, and for outputting a fourth output signal obtained by modulating the pulse width of the pulse wave by the third output signal; a low pass filter for smoothing the fourth output signal to output a fifth output signal; a VCO circuit the control voltage of which is the fifth output signal; a first frequency divider circuit for frequency dividing an output signal of the VCO circuit by N to output a system clock signal; and a second frequency divider circuit for frequency dividing, by M, and feeding the system clock signal back to the frequency and phase comparator.

### TECHNICAL FIELD

The present invention relates to a system clock generation circuit and, more particularly, to a digitized system clock generation circuit that can reproduce data of a wobble signal at CAV (constant angular velocity), has less jitters in the obtained system clock signal, and can generate a stable system clock signal in the case of losses of the wobble signal.

### BACKGROUND TECHNOLOGY

To record write data on an optical disk, such as CD-R/RW or DVD-RAM, data writing is carried out by EFM-modulating the write data and irradiating them to a predetermined track of the optical disk, using a laser beam controlled for writing by a laser controller.

On an optical disk like this, grooves are formed in a wobbling manner so that synchronizing signal for rotation control and address information (absolute time information) are recorded as wobble signals.

A wobble signal is a signal which is FSK-modulated with a modulation signal (BIDATA) of biphase code, and when the disk rotation is at a specified linear speed, the wobble frequency fWBL is 22.05±1 kHz (at 1-time speed reproduction).

ATIP (Absolute Time In Pregroove) signals containing absolute time information which is data-reproduced from wobble signals are comprised of a synchronizing signal, address data (absolute time data) and an error detection code CRC as a BIDATA, and normally come in units of 42 bits.

And the repetition frequency of the synchronizing signal is 75 Hz. Reproduction of such data recorded as wobble signals on an optical disk requires a demodulation circuit for demodulating the data of the wobble signals.

Known as this type of system clock generation circuit is one described in Patent Document 1.

### [Patent Document 1]

Japanese Patent Application Laid-Open No. 2001-143404.

FIG. 5 is an illustration showing an outline structure of a system clock generation circuit in synchronism with the wobble signals as introduced in the above-mentioned Patent Document 1 and the like.

The system clock generation circuit shown in FIG. 5 is structured as a PLL circuit and thus operates in such a manner that the wobble signal WBL detected from an optical disk locks to the system clock signal WPCLK. It is comprised of a phase comparison circuit 10, a speed (frequency) comparison circuit 20, charge-pump circuits 30 and 40, a low-pass filter (LPF) 50, a voltage control oscillator (VCO) 60 and an N division circuit 70 (N being an integer).

The synchronizing signal and ATIP signal are detected by inputting the system clock (WPCLK) generated by this PLL system clock generation circuit to a not-shown wobble signal FM demodulation circuit or digital PLL (DTLL).

To perform a data recording by CAV-driving an optical disk, a spindle motor driving the optical disk is driven at a constant speed.

To explain here on the assumption that the constant rotational speed is a specified speed, namely, a 1-time speed, then the wobble frequency fWBL at the inner circumferential area of the tracks of the optical disk will be 22.05±1 kHz.

On the outer side of the inner circumferential tracks, the wobble frequency fWBL will take a frequency higher than 22.05±1 kHz. In this manner, the range of frequency change of the wobble frequency fWBL is, for instance, about 22 kHz to about 53 kHz. The wobble signal WBL is inputted to the input terminal A of one of the phase comparison circuit 10 and the speed (frequency) comparison circuit 20. To the other input terminal B, the output of the VCO 60 is inputted after it is N-frequency-divided by the frequency divider 70.

The phase comparison circuit 10 outputs a charge-up signal, which goes high during a period corresponding to the phase difference between the rising edge of an input pulse to the input terminal A and the rising edge of an input pulse to the input terminal B, and sends it out to the charge-pump circuit 30 via the inverted buffer amplifier 31.

The phase comparison circuit 10 sends out a charge-down signal, which goes high during a period corresponding to the phase difference between the rising edge of an input pulse to the input terminal B and the rising edge of an input pulse to the input terminal A, to the charge-pump circuit 30.

Similarly, the speed comparison circuit 20 also prepares a signal based on the difference in speed (frequency), supplies a charge-up signal to a p-channel transistor 43 via an inverted buffer amplifier 41, and supplies a charge-down signal to an n-channel transistor 44. The charge-pump circuit 30 is comprised of an inverted buffer amplifier 31, a constant current source 32, a p-channel transistor 33, an n-channel transistor 34, and a constant current source 35.

The charge-pump circuit 40 is comprised of an inverted buffer amplifier 41, a constant current source 42, a p-channel transistor 43, an n-channel transistor 44, and a constant current source 45.

Based on the charge-up signal from the phase comparator 10, a constant current I0 is supplied to the low-pass filter 50, and based on the charge-down signal, a constant current I0 is sucked out as a sink current from the low-pass filter 50 to the charge-pump circuit 30. Similarly, based on the charge-up signal from the speed comparison circuit 20, a constant current I1 is supplied to the low-pass filter 50, and based on the charge-down signal, a constant current I1 is sucked out as a sink current to the charge-pump circuit 40. The low-pass filter (LPF) 50 is comprised of a resistance R and capacitances C1 and C2; the potential on the signal line 51 changes with the inflow of charge-up current and outflow of charge-down current; and a smoothed voltage is supplied as the control voltage of the VCO 60. The VCO 60 outputs an oscillation output signal of a frequency that allows following-up of the wobble signal WBL in response to the control voltage.

This sets a PLL loop control state as the 1/N frequency-divided signal is fed back and supplied to the phase comparison circuit 10 and the speed comparison circuit 20. As a result, the wobble signal WBL and the system clock signal WPCLK are locked to each other.

A system clock generation circuit as shown in FIG. 5 requires two comparison circuits, namely, a phase comparison circuit 10 for phase comparison and a speed comparison circuit 20 for speed (frequency) comparison. Also, if the system clock WPCLK is to be operated in a wide range of 1-time speed to 56-time speed, then it will be necessary to change the value of constant current I0 and I1 or resistance R.

To change the analog values of these I0, I1, and R, it is necessary to mount a separate analog circuit therefor, and this creates a problem of a large circuit mounting area required. Moreover, it is necessary to provide two capacitances C1 and C2 to be externally attached to the low-pass filter circuit 50.

Thus, there has been a problem that the system clock generation circuit used in a conventional data demodulation circuit tends to have a larger analog circuit mounting area and, when a single-chip integrated circuit is created, the chip area tends to be large. Also, there has been another problem that the use of two externally-attached capacitances makes the adjustment more complex.

### DISCLOSURE OF THE INVENTION

The present invention has been made to solve the above-mentioned problems, and an object thereof is to provide a data demodulation circuit for wobble signals, which can reduce the circuit scale by reducing the number of externally-attached capacitances to make a low-pass filter of simpler structure and at the same time without the use of a charge-pump circuit.

A system clock generation circuit according to the present invention is a system clock generation circuit which generates a system clock signal locked to a wobble signal, based on a wobble signal retrieved from an optical disk rotated at a constant angular velocity (CAV), so as to perform a CAV record on the optical disk, and the circuit comprises: a first PLL circuit which clocks the wobble signal and a first reference clock signal in frequency and phase; a frequency phase comparator which compares a first output signal from the first PLL circuit with the system clock signal and which outputs a second output signal based on a frequency difference and a phase difference; a PLL filter which outputs a third output signal by effecting a predetermined cutoff upon the second output signal; a pulse-width modulation circuit which generates a pulse wave having a second reference clock signal as a carrier frequency and which outputs a fourth output signal whose pulse width of the pulse wave has been modulated by the third output signal; a low-pass filter which smoothes the fourth output signal by effecting a predetermined cutoff thereupon so as to output a fifth signal; a VCO circuit which generates a sixth output signal having an oscillation frequency of a predetermined frequency range wherein the fifth output signal serves as a control voltage; a first frequency dividing circuit which outputs the system clock signal by N frequency dividing the sixth output signal wherein N is an integer; and a second frequency dividing circuit which M frequency-divides the system clock signal and then supplies it in feedback to the frequency phase comparator wherein M is an integer.

In a system clock generation circuit according to the present invention, a frequency division ratio of the second frequency dividing circuit is varied based on a phase difference between a sub-synchronizing signal (SUBsync) outputted from an encoder for CAV record information and an ATIP synchronizing signal (ATIPsync) derived from the wobble signal, and the ATIP synchronizing signal is locked to the sub-synchronizing signal.

In a system clock generation circuit according to the present invention, there is provided a selection circuit which inputs selectively the wobble signal or a third reference clock signal to the first PLL circuit.

In a system clock generation circuit according to the present invention, the first PLL circuit is structured as a PI type digital filter.

In a system clock generation circuit according to the present invention, the third output signal is so divided as to minimize the variation within a cycle of the carrier frequency of the pulse-width modulation circuit and is supplied to the pulse-width modulation circuit.

For example, in a system clock generation circuit according to the present invention, a reference clock signal is selected in the neighborhood of 203 MHz and the reference clock signal is divided so as to acquire the first, the second and the third reference clock signal.

### THE BEST MODE FOR CARRYING OUT THE INVENTION

FIG. 1 is an illustration showing a data demodulation circuit for wobble signals according to an embodiment of the present invention.

Different from a conventional circuit structure as shown in FIG. 5, the circuit according to the present invention is so structured as to equivalently perform the same function as the charge-pump circuit by deriving a phase error by using a pulse-width modulation (PWM) circuit 111 without using a charge-pump circuit and changing the pulse width of the PWM circuit 111 based on the phase error signal.

A wobble signal WBL 302, at a CAV drive, is rotating at a constant angular velocity, and at 1-time speed, wobble signals WBL having FSK-modulated wobble frequencies fWBL of 22 kHz to 53 kHz are supplied to one of the terminals of a multiplexer 105. A third reference clock signal 304 is inputted to the other terminal of the multiplexer 105, and a third reference clock signal 304, of which the changing range of the wobble frequency fWBL at a CAV drive of 1-time speed is from 22 kHz to 53 kHz, is supplied to the multiplexer 105.

According to the present embodiment, an oscillation of 33.8688 MHz is produced by a crystal oscillator 101; this is sexenary-multiplied to get an oscillation of a reference signal having a frequency of 203.2128 MHz (about 203 MHz); and 33.8688 MHz is frequency-divided by a frequency dividing circuit 103 to use it as a third reference clock signal 304. It is to be noted that it is possible to selectively switch between the third reference clock signal 304 and the wobble signal (WBL) 302 by a selection signal 310. Thus, until a wobble signal WBL having a predetermined frequency is obtained from the optical disk, the third reference clock signal 304 is selected to generate the system clock WPCLK. And at the stage when the wobble signal WBL is available, the multiplexer 105 is switched by the selection signal 310 to effect the selection of the wobble signal.

The output from the multiplexer 105 is inputted to a PLL circuit 107 and locked in phase and frequency to the first reference clock signal 306 derived from the PLL circuit 107. When the multiplexer 105 has selected the wobble signal and, in addition, the PLL circuit 107 has phase-locked, the wobble signal and the PLL circuit 107 maintain a phase-lock state. It is to be noted that the first reference clock signal 306 can be supplied after a frequency division of the aforementioned signal of about 34 MHz. The output of the PLL 107 is supplied to one of the inputs of the frequency phase comparator (FPC) 109. To the other input of the FPC 109, a 1/M frequency-divided signal of the system clock signal WPCLK is inputted, and after a frequency and phase comparison there, an output signal based on the difference is supplied to a PLL filter 200.

The PLL filter 200 may be structured as a PI type digital filter. That is, it is comprised of multipliers 201 and 203 having the coefficients K0 and K1, adders 202 and 205, and a delay circuit 204.

The PLL filter 200, which gives a predetermined cutoff to the output from the FPC 109, can easily effect a change in cutoff frequency by changing the coefficients K0 and K1 of the multipliers 201 and 203.

The output signal from the PLL filter 200 is inputted to the PWM circuit 111. The PWM circuit 111 generates a pulse wave having the second reference clock signal 308 as the carrier frequency, and the pulse width of the pulse wave is modulated by the signal from the PLL filter 200. Note that the second reference clock signal 308 used in the present embodiment is a reference signal that has a frequency of 25.4 MHz after a ⅛ frequency division of the aforementioned about 203 MHz.

The output of the PWM circuit 111 is supplied to a low-pass filter 113 which is comprised of R and C. The low-pass filter 113 smoothes the output of the PWM circuit 111 by giving a predetermined cutoff and supplies a control voltage to the subsequent VCO 115.

For a low-pass filter 113 used in the present embodiment, the values of R and C are so selected as to give a cutoff of 10 kHz. The VCO 115 is so structured as to output an oscillation frequency having a frequency change of about 200 MHz in response to a 1-volt change of control voltage.

The output from the VCO 115 is N (N being an integer) frequency-divided by a frequency divider 117 and further M (M being an integer) frequency-divided by a frequency divider 119 before it is supplied in feedback to the FPC 109. Thereby, the FPC 109 makes a frequency- and phase-comparison between the output signal of the PLL 107 and the output signal from the frequency divider 119 and outputs a signal based on the difference therebetween.

Through a PLL operation by a closed loop like this, the system clock signal WPCLK is outputted as a signal whose frequency and phase are locked to the wobble signal WBL.

It is to be noted that the frequency division ratio N to be selected by the frequency divider 117 is 1, 2 or 4 in accordance with the rotation speed multiple of the optical disk. The frequency division ratio M of the frequency divider 119 is normally set at 686.

FIG. 2 shows a sub-synchronizing signal SUBsync outputted from an encoder for CAV record information and an ATIP synchronizing signal ATIPsync derived from the wobble signal WBL, and the sub-synchronizing signal outputs a synchronizing signal of 75 Hz when the optical disk is rotating at a 1-time speed.

The ATIP synchronizing signal is a signal read by a data demodulation circuit from the optical disk, and it is necessary that the leading edge be locked to the synchronizing signal within ±2 frames.

In the circuit according to the present invention, a phase difference between the sub-synchronizing signal 311 and the ATIP synchronizing signal 312 is detected by a phase difference comparison circuit 121. The frequency division ratio M of the frequency divider 119 is changed based on this detection value, so that the sub-synchronizing signal is brought into agreement with the ATIP synchronizing signal.

To be more concrete, this is realized by changing the frequency division ratio 686 to 688 or 684, for instance.

The thus prepared system clock WPCLK is used as a channel clock for CAV recording, and it can also be used as a channel clock for constant linear velocity (CLV) recording.

The loop gain of the CAVPLL can be adjusted easily by changing the coefficient K0 of the multiplier 201 of the PLL filter 200. Similarly, the coefficient K1 of the multiplier 203 determines the cutoff frequency of the PLL filter 200. Hence, the PLL loop can be easily stabilized by appropriately selecting the coefficients K0 and K1.

It is desirable that the data for changing the pulse width of the carrier frequency of the PWM circuit by the output from the PLL filter 200 be supplied after being so divided as to minimize the variation within a cycle of the carrier frequency. This is because supplying the data after so dividing it as to minimize the variation within a cycle makes it possible to acquire oscillation stably without significant variation in the control voltage given to the VCO 115.

FIG. 3 is an illustration showing an example in which an output voltage from the PLL filter is divided and supplied to the PWM circuit 111.

Through a control as described above, the standard deviation of the variation in the system clock signal WPCLK has been controlled to 1% or less. Also, a PWM carrier attenuation rate of −60 dB has been realized.

According to the present embodiment, a reference signal is generated by using a reference clock having a clock frequency of about 203 MHz; however, if the variation of the VCO 115 due to the carrier of the PWM circuit 111 is to be made smaller, it is necessary to raise the frequency of the carrier signal supplied to the PWM. Accordingly, it is preferable that the second reference clock signal 308 be set as high as practicable within the range of the drive frequency of a device.

FIG. 4 shows an output waveform of the VCO 60, which is indicative of a transition to a stable state in an extremely short time (about 150 μs).

### INDUSTRIAL APPLICABILITY

Hereinabove, a detailed description has been given of embodiments of the present invention, and according to the present invention, the use of a digital FPC combining a phase comparison circuit and a speed comparison circuit makes it possible to perform the frequency control and phase control simultaneously.

Also, the digital FPC can be easily comprised of two JK flip-flops and a gate, thus presenting an advantage of simplified circuit configuration.

Furthermore, since the PLL filter used is a digital PI filter, the cutoff frequency can be changed easily.

Also, the PWM circuit used in place of the charge-pump circuit can be structured as a counter, thus presenting an advantage of a simplified circuit.

Also, the dual PLL structure of the whole circuit makes it possible to acquire a clock signal stable against the loss of wobble signals.

### BRIEF DESCRIPTION OF THE DRAWINGS

FIG. 1 shows a data demodulation circuit for wobble signals according to an embodiment of the present invention.

FIG. 2 is an illustration showing a relation of phase difference between a sub-synchronizing signal outputted from an encoder and an ATIP synchronizing signal derived from a wobble signal.

FIG. 3 is an illustration showing an example Of signal inputted to a PWM circuit.

FIG. 4 shows a change in VCO.

FIG. 5 is an illustration showing an outline structure of a system clock generation circuit used in a data demodulation circuit of wobble signals as introduced in Patent Document 1 and the like.

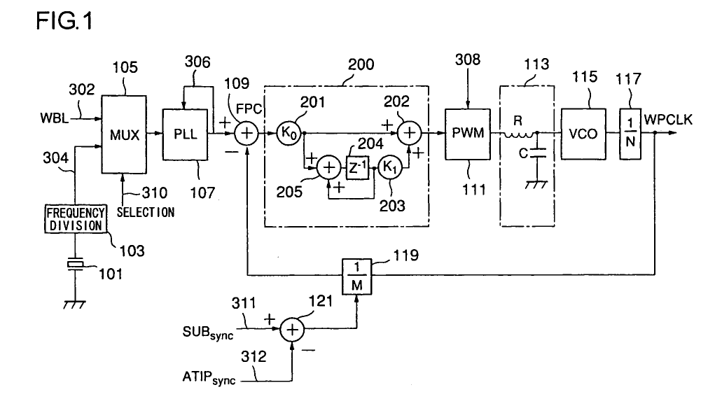

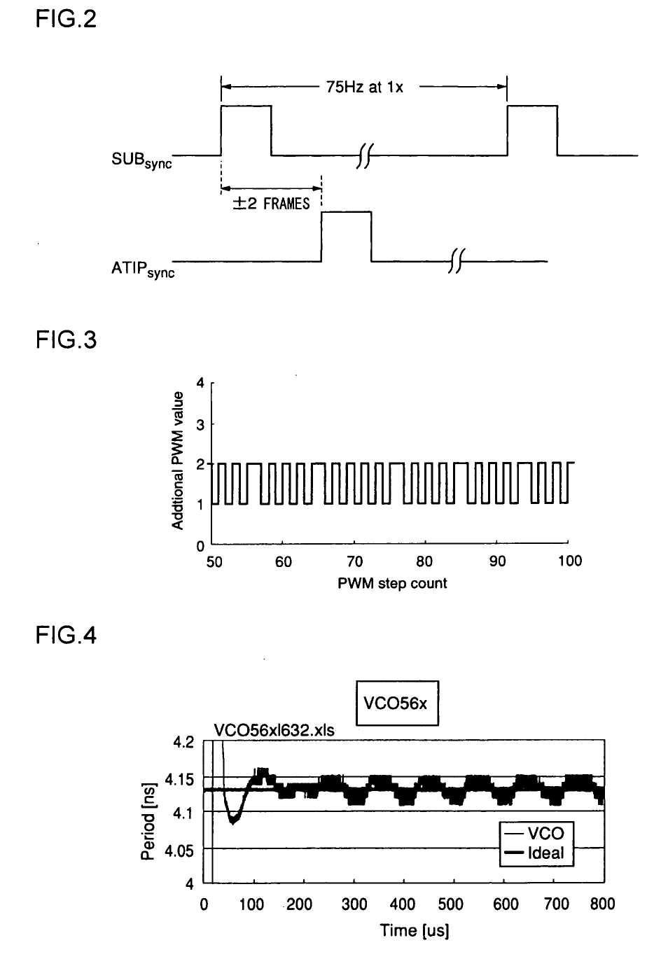

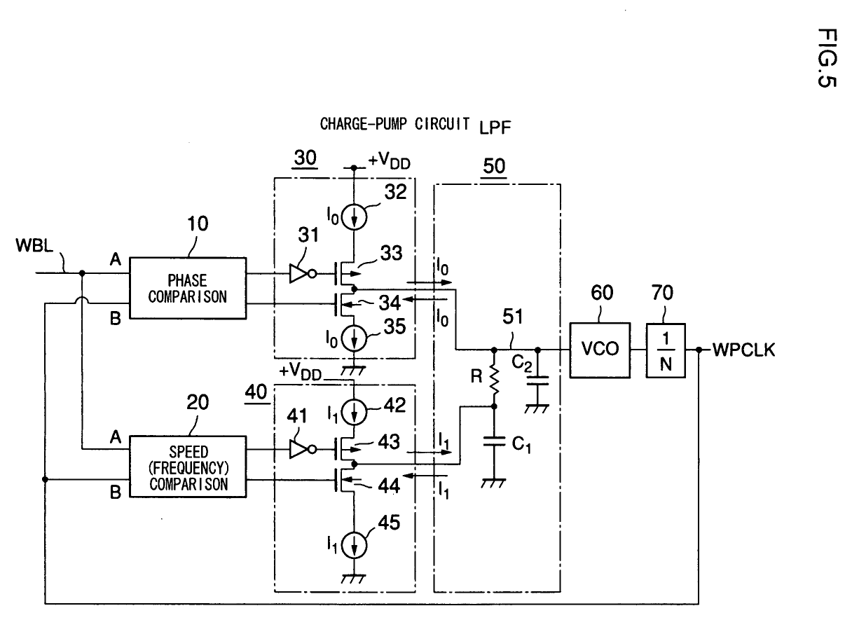

### DESCRIPTION OF REFERENCE NUMERALS

101 crystal oscillator, 103 frequency dividing circuit, 105 multiplexer, 107 PLL circuit, 109 frequency phase comparator (FPC), 111 pulse-width modulation (PWM) circuit, 113 low-pass filter, 115 VCO, 117, 119 frequency divider, 121 phase difference comparison circuit, 200 PLL filter, 201, 203 multiplier, 202, 205 adder, 204 delay circuit, 302 wobble signal WBL, 304, 306, 308 third, first, second reference signal, 310 sub-synchronizing signal, 312 ATIP synchronizing signal

1. A system clock generation circuit for generating a system clock signal locked to a wobble signal, based on a wobble signal retrieved from an optical disk rotated at a constant angular velocity (CAV), so as to perform a CAV record on the optical disk, the circuit comprising:
a first PLL circuit which locks the wobble signal and a first reference clock signal in frequency and phase;
a frequency phase comparator which compares a first output signal from said first PLL circuit with the system clock signal and which outputs a second output signal based on a frequency difference and a phase difference;
a PLL filter which outputs a third output signal by effecting a predetermined cutoff upon the second output signal;
a pulse-width modulation circuit which generates a pulse wave having a second reference clock signal as a carrier frequency and which outputs a fourth output signal whose pulse width of the pulse wave has been modulated by the third output signal;
a low-pass filter which smoothes the fourth output signal by effecting a predetermined cutoff thereupon so as to output a fifth signal;
a VCO circuit which generates a sixth output signal having an oscillation frequency of a predetermined frequency range wherein the fifth output signal serves as a control voltage;
a first frequency dividing circuit which outputs the system clock signal by N frequency dividing the sixth output signal wherein N is an integer; and
a second frequency dividing circuit which M frequency-divides the system clock signal and then supplies it in feedback to said frequency phase comparator wherein M is an integer.

2. A system clock generation circuit according to claim 1, wherein a frequency division ratio of said second frequency dividing circuit is varied based on a phase difference between a sub-synchronizing signal (SUBsync) outputted from an encoder for CAV record information and an ATIP synchronizing signal (ATIPsync) derived from the wobble signal, and the ATIP synchronizing signal is locked to the sub-synchronizing signal.

3. A system clock generation circuit according to claim 1, further comprising a selection circuit which inputs selectively the wobble signal or a third reference clock signal to said first PLL circuit.

4. A system clock generation circuit according to claim 1, wherein said PLL filter is structured as a PI type digital filter.

5. A system clock generation circuit according to claim 1, wherein the third output signal is so divided as to minimize the variation within a cycle of the carrier frequency of said pulse-width modulation circuit and is supplied to said pulse-width modulation circuit.

6. A system clock generation circuit according to claim 3, wherein a reference clock signal is selected in the neighborhood of 203 MHz and the reference clock signal is divided so as to acquire the first, the second and the third reference clock signal.
